(19) United States
(12) Patent Application Publication
(10) Pub. No.: US 2023/0154576 A1
(43) Pub. Date: May 18, 2023

(54) METHOD AND APPARATUS FOR TARGETING PEOPLE FOR PARTICIPATION IN CLINICAL TRIALS

(71) Applicant: Medical Meditation Corporation, Calgary (CA)

(72) Inventor: Nikhil JOSHI, Calgary (CA)

(73) Assignee: Medical Meditation Corporation, Calgary (CA)

(21) Appl. No.: 18/052,757

(22) Filed: Nov. 4, 2022

(30) Foreign Application Priority Data
Nov. 16, 2021 (CA) 3,139,286

(51) Int. Cl. G16H 10/20; H04W 4/029

(52) U.S. Cl. CPC G16H 10/20; H04W 4/029

(57) ABSTRACT

Disclosed is a method for execution by a computing device. The method involves receiving user input regarding an ailment, and responsive to the user input, playing media content relating to the ailment. The media content can for example include guided meditation for coping with the ailment. In accordance with an embodiment of the disclosure, the method also involves, upon an opportunity to participate in a clinical trial relating to the ailment, presenting details of the opportunity. In this manner, people can be targeted for participation in the clinical trial based on their consumption of media content. In some implementations, responsive to user input regarding interest to participate in the clinical trial, the method also involves transmitting a message over a network indicating that interest. This can enable details of interested parties to be compiled by a server.

### RELATED APPLICATION

This patent application claims priority to Canadian Patent application no. 3,139,286 filed on Nov. 16, 2021, the entire disclosure of which is incorporated by reference.

### FIELD OF THE DISCLOSURE

This disclosure relates to communication systems, and more particularly to communication systems for targeting people for participation in clinical trials.

### BACKGROUND

It can be difficult to recruit people for clinical trials. For example, by one estimate, about 85% of clinical trials go unfilled, and of the 15% of clinical trials that proceed, 80% of them are delayed due to delays in recruitment. Older methods that rely on advertising through old media such as radio and/or print adds are not very effective. Newer methods introduce online social media, but they are largely research-centric and not very effective at finding recruits. Note that many people are generally reluctant to participate in clinical trials because of the experimental nature of clinical trials. Without an effective way of recruiting, it is difficult to overcome that general reluctance.

Conventional approaches to recruit people for clinical trials leave much to be desired. It is desirable to improve upon the conventional approaches by employing technology to address or mitigate some or all of the aforementioned shortcomings.

### SUMMARY OF THE DISCLOSURE

Disclosed is a method for execution by a computing device. The method involves receiving user input regarding an ailment, and responsive to the user input, playing media content relating to the ailment. The media content can for example include guided meditation for coping with the ailment. In accordance with an embodiment of the disclosure, the method also involves, upon an opportunity to participate in a clinical trial relating to the ailment, presenting details of the opportunity. In this manner, people can be targeted for participation in the clinical trial based on their consumption of media content.

In some implementations, responsive to user input regarding interest to participate in the clinical trial, the method also involves transmitting a message over a network indicating that interest. This can enable details of interested parties to be compiled by a server.

Also disclosed is a non-transitory computer readable medium having recorded thereon an application having statements and instructions that, when executed by a processor of a computing device, configure the computing device to implement the method summarized above. In some implementations, the application is downloaded to the computing device from a server.

Also disclosed is a computing device having a network adapter, a non-transitory computer readable medium, and a processor coupled to the network adapter and the non-transitory computer readable medium. In accordance with an embodiment of the disclosure, the non-transitory computer readable medium has recorded thereon an application having statements and instructions that, when executed by the processor, configures the computing device to implement the method summarized above.

Also disclosed is a server having a network adapter, a non-transitory computer readable medium, and a processor coupled to the network adapter and the non-transitory computer readable medium. In accordance with an embodiment of the disclosure, the non-transitory computer readable medium has recorded thereon an application having statements and instructions that, when executed by a processor of a computing device, configure the computing device to implement the method as summarized above. In addition, the processor is configured to, upon receiving a request via the network adapter for the application, transmit a copy of the application via the network adapter. In some implementations, the processor is configured to, upon receiving messages via the network adapter indicating interest to participate in the clinical trial, compile details of that interest.

Also disclosed is a server having a network adapter, a non-transitory computer readable medium, and a processor coupled to the network adapter and the non-transitory computer readable medium. In accordance with an embodiment of the disclosure, the server is web server and the processor is configured to generate web content suitable for a web browser of a computing device based on data stored on the non-transitory computer readable medium, such that the web content enables the computing device to implement the method summarized above, and wherein the processor is configured to transmit the web content via the network adapter.

Other aspects and features of the present disclosure will become apparent, to those ordinarily skilled in the art, upon review of the following description of the various embodiments of the disclosure.

### BRIEF DESCRIPTION OF THE DRAWINGS

Embodiments will now be described with reference to the attached drawings in which:

FIG. 1 is a block diagram of an example communication system having a server coupled to a plurality of computing devices via a network; and

FIG. 2 is a flowchart of a method of targeting a person for participation in a clinical trial.

### DETAILED DESCRIPTION OF EMBODIMENTS

It should be understood at the outset that although illustrative implementations of one or more embodiments of the present disclosure are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

### Introduction

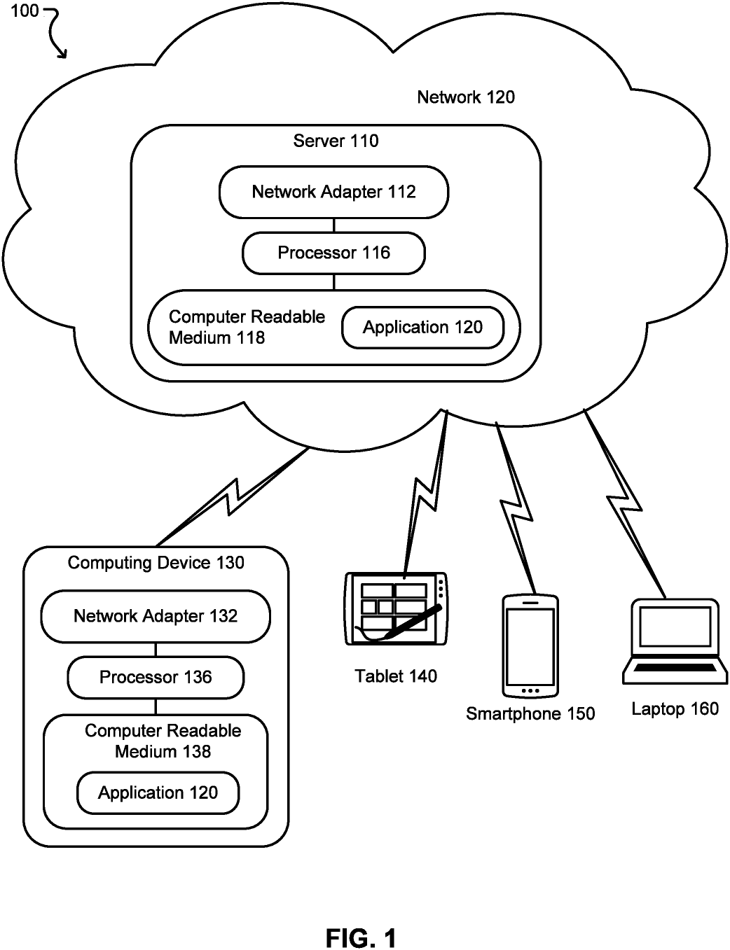

Referring first to FIG. 1, shown is a block diagram of an example communication system 100 having a server 110 coupled to a plurality of computing devices 130,140,150,160 via a network 120. The communication system 100 can have other components as well, but these are not shown for simplicity. The server 110 has a network adapter 112 for communicating with the computing devices 130,140,150,160 over the network 120, a processor 116, a computer readable medium 118, and may have other components that are not shown for simplicity. The computing device 130 has a network adapter 132 for communicating with the server 110 over the network 120, a processor 136, a computer readable medium 138, and may have other components that are not shown for simplicity. Details of the other computing devices 140,150,160 are not shown for simplicity, but they can have similar components as the computing device 130.

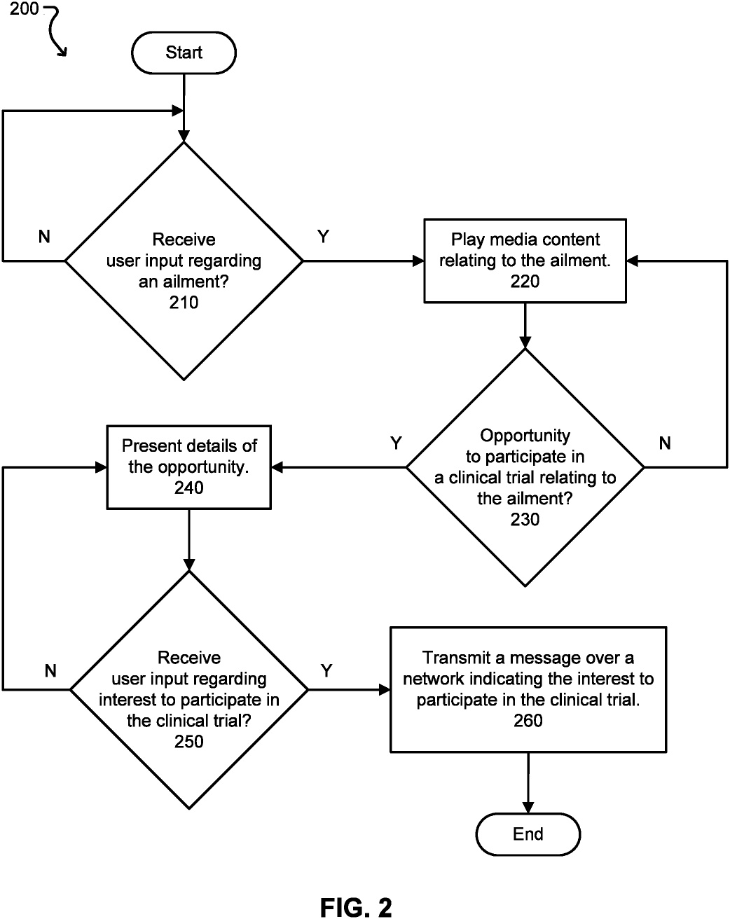

In the illustrated example, the server 110 has an application 120, and the computing devices 130,140,150,160 can download a copy of the application 120 over the network 120. Once downloaded, the application 120 can be executed. The operation of the computing device 130 when executing the application 120 will be described below with reference to FIG. 2, which is a flowchart of a method 200 of targeting a person for participation in a clinical trial. Although the method 200 of FIG. 2 is described below with reference to the computing device 130 in the communication system 100 shown in FIG. 1, it is to be understood that the method 200 of FIG. 2 is applicable to other communication systems. Also, it is to be understood that the downloading of an application is not essential. For instance, in other implementations, the server 110 is a web server and the computing device 130 interacts with the web interface through a web browse. Other implementations are possible.

If at step 210 the computing device 130 receives user input regarding an ailment, then at step 220 the computing device 130 plays media content relating to the ailment. The media content can for example include guided meditation for coping with the ailment. In accordance with an embodiment of the disclosure, if at step 230 there is an opportunity to participate in a clinical trial relating to the ailment, then at step 240 the computing device 130 presents details of the opportunity. In this manner, people can be targeted for participation in the clinical trial based on their consumption of media content. This is a practical application of computer technology that builds upon the consumption of media content and thus computer technology is part of the actual invention.

If at step 250 the computing device 130 receives user input regarding interest to participate in the clinical trial, then at step 260 the computing device 130 transmits a message over the network 120 indicating that interest. This can enable details of interested parties to be compiled by the server 110. In some implementations, such details of interested parties is compiled by the same server 110 that distributes copies of the application 110. In other implementations, such details of interested parties is compiled by another server. For instance, a first server can distribute copies of the application 120 while a second server can receive messages indicating interest to participate in the clinical trial. Other implementations are possible.

In other implementations, the computing device 130 provides contact information (e.g. phone number and/or email address) so that the user can express their interest to participate in the clinical trial through other means. In such implementations, the computing device 130 might not transmit a message indicating interest to participate in the clinical trial.

Once details of interested parties are compiled, they can be reviewed according to an inclusion criteria, submitted as a proposal for recruitment, and it may be possible to obtain a finders fee upon successful recruitment of the interested parties. Finders fees can be significant, for example 10% of the cost of a clinical trial which for example could be $25k per diabetes patient (~$2.5k finders fee) or $80k per cancer patient (~$8k finders fee).

In some implementations, the user is presented with the details of the opportunity only if the clinical trial is in a geographic location (e.g. city, province or state) associated with the user. In some implementations, the geographic location is inferred from a location from which the media content relating to the ailment has been promoted. For example, upon promoting the media content at a meeting in Ottawa, subsequent downloads of the application can be inferred to be for people living in Ottawa or surrounding areas. In such implementations, it is not necessary to determine or track a location of the user. In other implementations, the geographic location is determined based on a location of the computing device 130, for example by using GPS (Global Positioning System) technology or other means. In other implementations, the geographic location is determined based on user input specifying location. For example, the user can specify that they reside in Ottawa. Other implementations are possible.

In some implementations, the user input at step 210 includes an indication of the ailment. For example, the user can specify their ailment as being “arthritis” with a view to receiving guided meditation for addressing their arthritis. In some implementations, the media content relating to the ailment is one of many different media contents that relate to the ailment, and the user selects which media content to be played. In some implementations, there are many different media contents that relate to multiple different ailments, and the user selects which media content to be played. Other implementations are possible.

In some implementations, the media content is stored on the server 110, and responsive to the user input, the media content is downloaded prior to playing the same. In other implementations, the media content is stored on another server. For instance, a first server can distribute copies of the application 120 while a second server can provide the media content. Other implementations are possible.

There are many possibilities for the media content. In some implementations, the media content includes audio data. In some implementations, the audio data encodes an audio track mixed with words relating to guided meditation for the ailment. In some implementations, the media content also includes video data. In some implementations, the media content is interactive, such as video conferencing for example. Other implementations are possible.

There are many possibilities for the computing devices 130,140,150,160. The computing devices 130,140,150,160 can for example include a desktop computer 130, a tablet compute 140, a smartphone 150, a laptop 160, and/or any other appropriate computing device. The computing devices 130,140,150,160 can communicate with the server 110 using wireless connections as depicted and/or wired connections. Although only four computing devices 130,140,150,160 are depicted, it is to be understood that there can be more or less than four computing devices, and typically there would be numerous computing devices.

There are many possibilities for the network 120. The network 120 can include several different networks even though such details are not shown for simplicity. For example, the network 120 can include a RAN (Radio Access Network) for communicating with wireless stations and the Internet for communicating with numerous other computing devices. The network 120 can have other components as well, but these details are not shown for simplicity.

There are many possibilities for the server 110. In some implementations, the server 110 includes an application server for distributing copies of the application 120. Additionally, or alternatively, the server 110 can include a web server for producing web content suitable for a web browser. In some implementations, the server 110 includes multiple servers to divide up functionality as noted above. Other implementations are possible.

There are many possibilities for the network adapter 112 of the server 110. In some implementations, the network adapter 112 is a single network adapter 112. In other implementations, the network adapter 112 includes multiple network adapters, for example a first network adapter for communicating with the one or more computing devices 130,140,150,160, and a second network adapter for communicating with other computing devices, such as computing devices utilized by a system administrator. Both wireless and wired network adapters are possible. Any suitable network adapter that can communicate via the network 120 is possible.

Although embodiments disclosed herein focus on software implementations, it is noted that other implementations are possible. It is noted that other implementations can include additional or alternative hardware components, such as any appropriately configured FPGA (Field-Programmable Gate Array), ASIC (Application-Specific Integrated Circuit), and/or microcontroller, for example. Functionality described herein could be implemented with any suitable combination of hardware, software and/or firmware.

According to another embodiment of the disclosure, there is provided a non-transitory computer readable medium having recorded thereon statements and instructions that, when executed by the processor 136 of the computing device 130, implement a method as described herein. The non-transitory computer readable medium can be the computer readable medium 118 of the server 110 shown in FIG. 1, the computer readable medium 138 of the computing device 130 shown in FIG. 1, or some other non-transitory computer readable medium. The non-transitory computer readable medium can for example include an SSD (Solid State Drive), a hard disk drive, a CD (Compact Disc), a DVD (Digital Video Disc), a BD (Blu-ray Disc), a memory stick, or any appropriate combination thereof.

Numerous modifications and variations of the present disclosure are possible in light of the above teachings. It is therefore to be understood that within the scope of the appended claims, the disclosure may be practised otherwise than as specifically described herein.

We claim:

1. A method for execution by a computing device, comprising:
receiving user input regarding an ailment;
responsive to the user input, playing media content relating to the ailment; and
upon an opportunity to participate in a clinical trial relating to the ailment, presenting details of the opportunity.

2. The method of claim 1, wherein the user input regarding the ailment is first user input and the method further comprises:
upon presenting the details of the opportunity to participate in the clinical trial, receiving second user input regarding interest to participate in the clinical trial; and
responsive to the second user input, transmitting a message over a network indicating the interest to participate in the clinical trial.

3. The method of claim 1, wherein the user input is from a user associated with a geographic region, and the method comprises:
if the opportunity to participate in the clinical trial is in the geographic location associated with the user, downloading the details of the opportunity prior to presenting the details of the opportunity, such that the user is presented with the details of the opportunity only if the clinical trial is in the geographic location associated with the user.

4. The method of claim 3, wherein the geographic location is inferred from a location from which the ailment and/or the media content relating to the ailment has been promoted.

5. The method of claim 3, further comprising:
determining the geographic location based on a location of the computing device.

6. The method of claim 3, further comprising:
determining the geographic location based on user input specifying location.

7. The method of claim 1, wherein the user input regarding the ailment comprises an indication of the ailment.

8. The method of claim 1, wherein:
the media content relating to the ailment is one of many different media contents that relate to the ailment; and
the user input regarding the ailment comprises a selection among the many different media contents.

9. The method of claim 1, wherein:
the media content relating to the ailment is one of many different media contents that relate to multiple different ailments;
the user input regarding the ailment comprises a selection among the many different media contents.

10. The method of claim 1, wherein the media content is stored on a server, and the method further comprises:
responsive to the user input, downloading the media content prior to playing the media content.

11. The method of claim 1, wherein media content comprises audio data.

12. The method of claim 11, wherein the audio data encodes an audio track mixed with words relating to guided meditation for the ailment.

13. The method of claim 1, further comprising:
downloading and executing an application;
wherein the media content is played by the application and the details of the opportunity are presented by the application.

14. A non-transitory computer readable medium having recorded thereon an application having statements and instructions that, when executed by a processor of a computing device, configure the computing device to implement the method of claim 1.

15. A computing device, comprising:
a network adapter;
a non-transitory computer readable medium; and
a processor coupled to the network adapter and the non-transitory computer readable medium;
wherein the non-transitory computer readable medium has recorded thereon an application having statements and instructions that, when executed by the processor, configures the computing device to implement the method of claim 1.

16. A server comprising:
a network adapter;
a non-transitory computer readable medium; and
a processor coupled to the network adapter and the non-transitory computer readable medium; wherein:
the non-transitory computer readable medium has recorded thereon an application having statements and instructions that, when executed by a processor of a computing device, configure the computing device to implement the method of claim 1; and
the processor of the server is configured to, upon receiving a request via the network adapter for the application, transmitting a copy of the application via the network adapter.

17. The server of claim 16, wherein:
the processor is configured to, upon receiving messages via the network adapter indicating interest to participate in the clinical trial, compiling details of that interest.

18. The server of claim 17, wherein the server comprises a first server for distributing the application and a second server for receiving the messages indicating interest to participate in the clinical trial.

19. A server, comprising:
a network adapter;
a non-transitory computer readable medium; and
a processor coupled to the network adapter and the non-transitory computer readable medium;
wherein the server is web server and the processor is configured to generate web content suitable for a web browser of a computing device based on data stored on the non-transitory computer readable medium, such that the web content enables the computing device to implement the method of claim 1, and wherein the processor is configured to transmit the web content via the network adapter.

20. The server of claim 19, wherein:
the processor is configured to, upon receiving messages via the network adapter indicating interest to participate in the clinical trial, compiling details of that interest.
